(19) United States
(12) Patent Application Publication
(10) Pub. No.: US 2024/0065730 A1
(43) Pub. Date: Feb. 29, 2024

(54) IMPROVEMENT IN DIRECT HAIR IMPLANTATION AUTOMATIC TRANSFER SERVICE

(71) Applicant: Lütfi KECICI, Istanbul (TR)

(72) Inventor: Lütfi KECICI, Istanbul (TR)

(21) Appl. No.: 18/275,225

(22) PCT Filed: Oct. 22, 2021
(86) PCT No.: PCT/TR2021/051088

§ 371 Date: Aug. 1, 2023

(30) Foreign Application Priority Data
Feb. 5, 2021 (TR) 2021/01749

(51) Int. Cl. A61B 17/34; A61B 34/30

(52) U.S. Cl. CPC A61B 17/3468; A61B 34/30; A61B 2017/00752

(57) ABSTRACT

A direct hair implantation automatic transfer device enables the quick transfer of the pens used in the DHI (Direct Hair Implantation) for procedures of hair implantation, eyebrow implantation, and beard implantation. The device includes a conveyor assembly including two empty pen bands positioned parallel to one another in the direction of z-axis and a full pen band positioned between the empty pen bands in a way making a certain angle with respect to the z-axis direction; an empty pen funnel and a full pen funnel, the lower and upper portions of which are formed by holes and which have a conical or plain cylindrical form with respect to the vertical centerline; and a robotic arm including at least one joint being associated in an engaging manner and having the freedom of motion with respect to one another and a sensitive gripper carrying the pen (K) at the tip portion thereof.

### CROSS REFERENCE TO THE RELATED APPLICATIONS

This application is the national phase entry of International Application No. PCT/TR2021/051088, filed on Oct. 22, 2021, which is based upon and claims priority to Turkish Patent Application No. 2021/01749, filed on Feb. 5, 2021, the entire contents of which are incorporated herein by reference.

### TECHNICAL FIELD

The present invention relates to an improvement made in the direct hair implantation automatic transfer device, enabling the quick transfer of the pens used in the DHI (Direct Hair Implantation) for the procedures of hair implantation, eyebrow implantation, beard implantation and all kinds of graft follicle transplantation.

### BACKGROUND

The hair implantation is a procedure for performing the permanent hair transplantation to the areas with the hair loss of the individuals who experience partial or complete baldness resulting from the hair loss whether for genetic or other metabolic and structural causes. Until the present, numerous techniques have been employed for this purpose. Whereas the undesirable conditions like inadequate hair follicle removal, scar development and unnatural appearance were encountered in the early hair implantations, the hair implantation techniques have at the present undergone the required innovations and improvements parallel with the developing technology. This innovative approach for the hair implantation brings about the advantages such as the prevention of the scars, the transplantation of an as great as possible number of hair follicles and the achievement of a natural appearance.

The hair implantation procedure comprises various steps. These steps broadly include the pre-intervention, intra-intervention and post-intervention steps. The hair implantation procedure in itself consists of 3 steps. These are removing the hair follicles, making the channels for placing the follicles and implanting the follicles. The hair follicles to be implanted for the purpose of hair implantation are obtained from the same individual, especially from the scalp on the back part of the head. In the cases where it is deemed appropriate, the follicles removed from the back or chest area may also be used. There are two main methods in existence for the removal of the hair follicles. These are the methods of Follicular Unit Transplantation (FUT) and Follicular Unit Extraction (FUE). In the FUT technique, a part of the scalp on the back of the head of the patient is removed in the form of a strip. The size of this strip is determined by the number of grafts needed. A small cut mark remains after the implantation. The obtained strips are divided into grafts containing 1-4 hair follicles and are rendered ready for implantation. The FUE technique is a method based on removing the individual grafts with a diameter of 0.9-0.5 mm again from the same area with the help of an electric micro-motor. Each of these grafts includes 1-4 hair follicles.

The hair implantation pens are available for use in the hair implantation procedure. The hair implantations performed by the use of a hair implantation pen involve the hair follicle that is positioned in the tip portion of the pen for being injected to the target area. When carrying out the hair implantation, the surgeon injects the hair follicle in the tip of the pen to the target area. It is necessary every time to dip the pen in a sterile liquid and then fix another hair follicle to the tip portion. These procedures are carried out by the assistants of the surgeon. The surgeon faces the necessity to continuously change position in order to receive the pens to the tip of which the hair follicle is fixed. Consequently, the joint pains develop in the surgeon and the surgeon assistants and affect their health adversely. This at the same time negatively affects the speed of the hair implantation procedure. The numbers of grafts and follicles removed are less in today's hair implantation procedures. The scar tissue forms during the hair implantation procedure. The density of grafts per centimeter is small. The removed grafts remain outside for a prolonged time and the tissue death occurs, as a result of the hair implantation not being performed quickly. Moreover, the hair implantation may be performed only with a single individual.

The patent application no. CN1146716A discloses “Apparatus for implanting hair roots, and means for placing hair roots on an assembling strip for use in the apparatus”. A system for hair transplantation comprises a pistol for rapid shooting of hair grafts into skin from an assembling strip with receptacles which have been loaded beforehand with such hair grafts. The system also comprises a means for placing such grafts in receptacles on the assembling strip, and said means includes a frame for a piece of skin which has been surgically cut out, a videocamera for detailed viewing of the skin piece as well as a computer for analysis of the video picture and control of a number of motors for moving and adjusting the assembling strip and drill rod for cutting out grafts from the skin piece. The frame also comprises a cooling device for cooling the skin piece.

The patent application no. WO2015174645A1 discloses “Automated Hair Transplant Device”. The present invention relates to an automated hair transplant device for transplanting a selected hair into a hair loss area. The transplanting method comprises: selecting a hair of which the root is healthy, making the selected hair have a predetermined length without harming the follicle thereof, putting the hair of a predetermined length in a hair transplant needle, inserting the hair transplant needle into the scalp into which the hair is transplanted, and then separating the hair transplant needle from the scalp while leaving the hair therein.

The patent application no. US2015133962A1 discloses “Systems and Methods for Harvesting, Storing, and Implanting Hair Grafts”. A system and method for harvesting, storing, and implanting biological units, in particular hair follicular units (FUs). The system is particularly useful to facilitate hair transplant procedures. The system may comprise an implanting tool, a cartridge with a plurality of receptacles for biological units and one or more control mechanisms. The device and cartridge for storing biological units may be incorporated within an overall automated or robotic system, or may form part of a semi-automated or even manual apparatus.

The above-described patent applications disclose the methods and devices employed in the hair implantation. The described systems, though being innovations in the art of hair implantation, are not able to become the systems that would facilitate the hair implantation by the desirable extent. The described products are unable to enable the hair implantation to be performed in a quick manner and further cause the surgeon to continuously change position. Moreover, said products are costly. Occupational accidents occur, resulting from the use of tools while performing the pen transfer during the hair implantation.

### SUMMARY

The present invention is an improvement made in the direct hair implantation automatic transfer device capable of overcoming the above-mentioned disadvantages characterized in that it is a novel technology, which is able to perform 4000 to 7000 graft transplants in a single session, is able to reduce the occupational accidents associated with the materials such as pen, etc., does not require the patients to visit again for a second session, is able to count the grafts transplanted, is compatible with the cold chain of the graft storage area, provides the possibility to perform the graft transplantation to the same patient by two surgeons, prevents the death of the graft tissues owing to the permanently maintained contact with sterile water, minimizes the material deformation resulting from the dropping of the hair implantation pens, enables the sterilization of the hair implantation pen by means of the UV light, has a protective transparent cover, enables the patient to have a live view of the procedure being performed, has a laser light system with the ability of calculating the number of grafts per area, is able to provide a paper slip containing the patient information-procedure information and is of low cost.

According to the systems disclosed in the applicant's inventions no. TR201805012 and TR201812047, the empty and full pens circulate between the surgeon and the nurse via a single band. However, the undesirable situations are observed to develop in such systems. A problem is encountered when the pens are adhered manually on the band with said band moving at a certain speed. Our older surgeons who face vision problems or who have unsteady hands are not able to position the pens on the band in an upright manner. This causes the pens to become placed at the undesired positions (in a horizontal or angular arrangement) on the band, and as a result, faults may occur in the manner of operation of the system or in the mechanical parts. Further, the hair follicles are unable to enter the sterilization chamber or the damage may be caused in the hair follicles.

Owing to the funnels included in our system according to the invention, the surgeon or the nurse is able to drop the pens in a direct manner into a funnel and it is in turn made possible to transfer the pens from the funnel onto the band and fix the same on the band, without encountering any problem of focusing in the placement of the pens on the band.

Another advantage of our system according to the invention is that the bands designated for the outgoing and incoming pens are separated from each other, thereby making certain on which band the empty and full pens are to advance and providing the ease of use for the surgeon or nurses who are new to the use of the invention. In addition, the empty pen band is divided into two opposing parts, making it possible to convey from both sides the pens to the location of the nurse. By this means, a quick filling process is achieved. The likelihood of confusing the full and empty pens, as a result of the pens under the control of the surgeon or the nurse escaping their attention, is zeroed out and the apparatus that provide this function in the existing methods are not needed.

Another advantage of our system according to the invention is that it is possible to perform the hair implantation procedures in an automated manner by means of a robotic arm. Depending on the patient, the hair implantation procedure may be performed successfully either automatically or manually (only by the surgeon). The position sensors placed on the head of the patient and the sensors on the robotic arm match one another and it is made possible to enable the control of the robotic arm by the software based on the signals received.

Owing to the ability to easily fix together the component parts of the invention, the installation of the system becomes easy and the costs are accordingly lower owing to a shorter assembly time. The system according to the system also has a robust structure.

### BRIEF DESCRIPTION OF THE DRAWINGS

The invention will be described with reference to the attached figures in order to enable the features of the invention to be more clearly understood and appreciated; however, it is not intended to limit the invention to these particular embodiments. On the contrary, it is aimed to include all the alternatives, modifications and equivalents in the scope of the invention as defined by the attached claims. It should be understood that the illustrated details are provided only for the purpose of describing the preferred embodiments of the present invention and of providing the most convenient and most easily understandable description of the structuring of the methods as well as the principles and conceptual features of the invention. In said drawings;

FIG. 1 is the view showing the working mode of the conveyor belt system belonging to the state of the art.

FIG. 2 is the perspective view of the system of the invention.

FIG. 3 is the cross-sectional view of the system of the invention.

FIG. 4 is the perspective view of the conveyor assembly of the system of the invention.

FIG. 5 is the perspective view of the robotic arm of the system of the invention.

The components in the figures to aid in an understanding of the present invention are assigned reference numerals as can be seen in the attached drawings and said reference numerals and the corresponding components are listed below.

### DESCRIPTION OF THE REFERENCE NUMERALS

1. Conveyor chassis
2. Transparent cover
3. Hair implantation pen holder
4. Motor
5. Pen collection apparatus
6. Sterile liquid chamber
7. Display
8. Empty pen funnel
9. Full pen funnel
10. Conveyor assembly
   10.1. Full pen band
   10.2. Empty pen band
   10.3. Gear
   10.4. Bearing
   10.5. Shaft
11. Robotic arm
   11.1. Joint
      11.1.1. Male part
      11.1.2. Female part
   11.2. Sensitive gripper
K. Pen

### DETAILED DESCRIPTION OF THE EMBODIMENTS

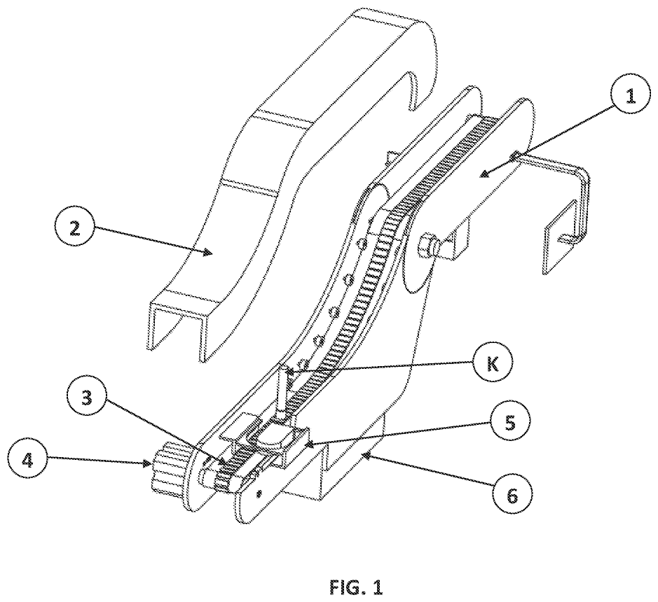

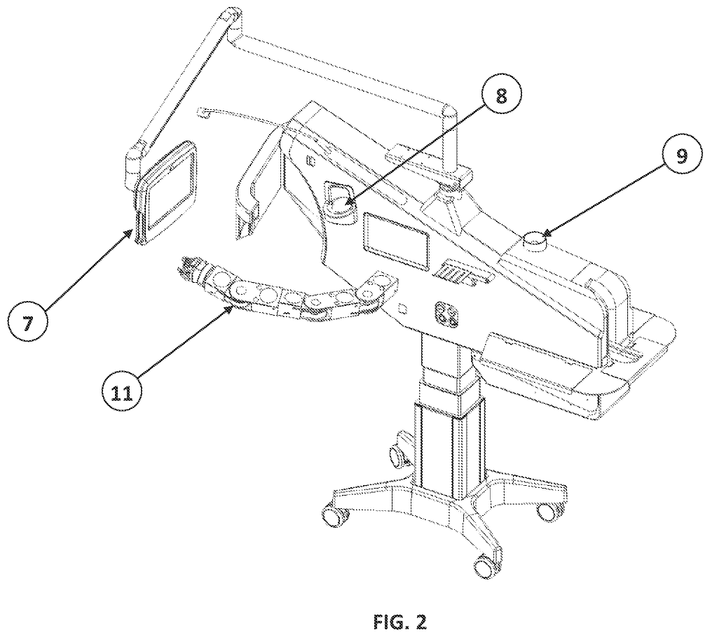

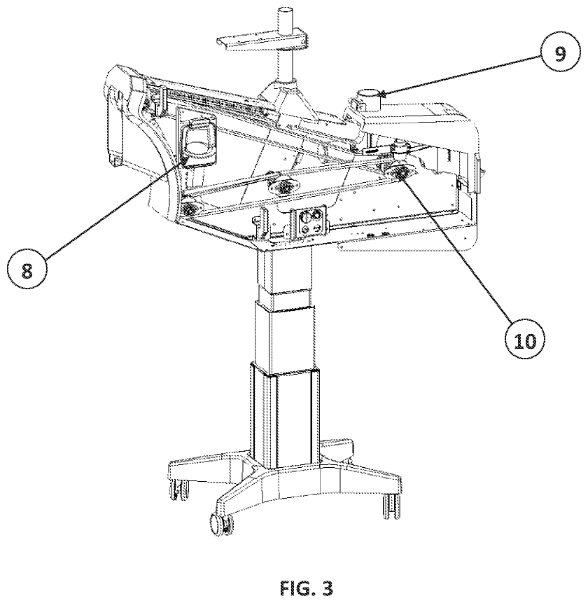

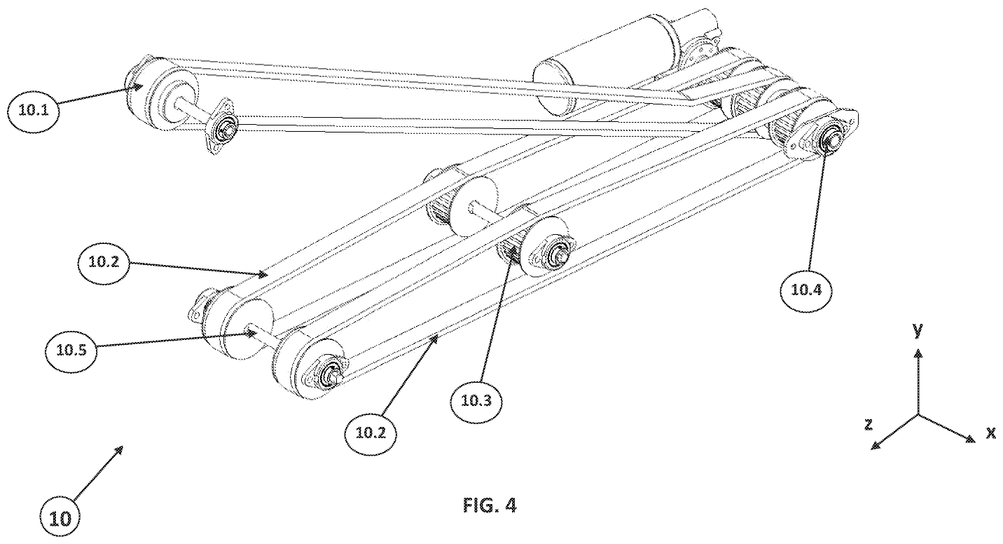

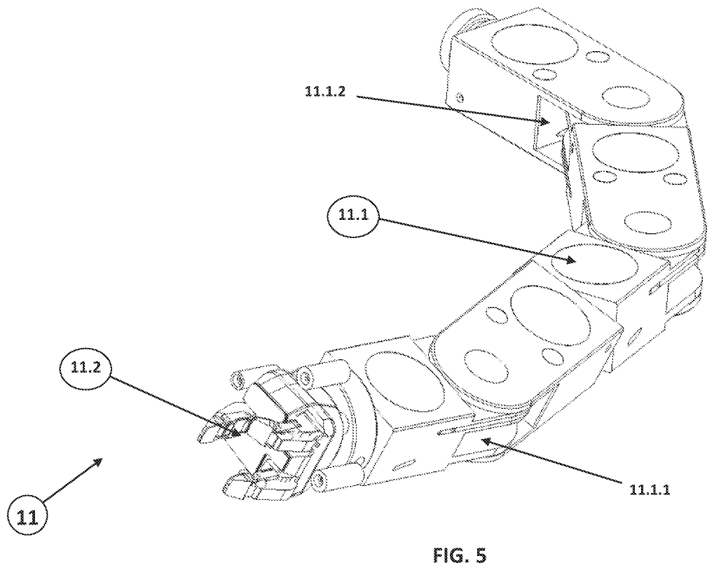

The invention comprises a conveyor assembly (10) including at least one empty pen band (10.2) positioned parallel to one another in the direction of z-axis and at least one full pen band (10.1) positioned between the empty pen bands (10.2) in a way making a certain angle with respect to the z-axis direction; an empty pen funnel (8) and a full pen funnel (9), the lower and upper portions of which are formed by holes and which have a conical or plain cylindrical form with respect to the vertical centerline; and a robotic arm (11) including at least one joint (11.1) being associated in an engaging manner and having the freedom of motion with respect to one another and a sensitive gripper (11.2) carrying the pen (K) at the tip portion thereof {FIG. 1, FIG. 2, FIG. 3, FIG. 4, FIG. 5}.

The conveyor assembly (10) disclosed in the invention comprises a full pen band (10.1) and an empty pen band (10.2) that move in opposite directions. The conveyor assembly (10) according to the invention has at least one gear (10.3) providing support for the movement of the bands. The conveyor assembly (10) according to the invention contains at least one shaft (10.5) that enables the bearing for the gears (10.3). The conveyor assembly (10) according to the invention has at least one bearing (10.4) that balances the loads imparted on the shafts (10.5) (FIG. 4).

The system according to the invention has an empty pen funnel (8), which is associated with the body in a way to deliver the pens (K) onto the empty pen band (10.2), and a full pen funnel (9), which is associated with the body in a way to deliver the pens (K) onto the full pen band (10.1) {FIG. 2, FIG. 3}.

The robotic arm (11) included in the invention comprises the joints (11.1) that enable the forward-backward, rightward-leftward, upward-downward and angular movements of the sensitive gripper (11.2). The robotic arm (11) included in the invention has at least one servo motor or step motor providing the movement torque to the joints (11.1). The robotic arm (11) included in the invention contains at least one position sensor located on the sensitive gripper (11.2). The engagement-type association between the joints (11.1) according to the invention is provided by the male part (11.1.1) and the female part (11.1.2) {FIG. 5}.

### DETAILED DESCRIPTION OF THE INVENTION

The system according to the invention basically comprises a transparent cover (2), a hair implantation pen holder (3), a motor (4), a pen collection apparatus (5), a sterile liquid chamber (6), a display (7), an empty pen funnel (8), a full pen funnel (9), a conveyor assembly (10) and a robotic arm (11). The conveyor assembly (10) comprises the components of the full pen band (10.1), empty pen band (10.2), gear (10.3), bearing (10.4) and shaft (10.5). The robotic arm (11) is formed by the components of the joint (11.1) and the sensitive gripper (11.2). The relationship between the joints (11.1) is provided by means of the male part (11.1.1) and the female part (11.1.2) {FIG. 1, FIG. 2, FIG. 3, FIG. 4, FIG. 5}.

The components of conveyor chassis (1), transparent cover (2), hair implantation pen holder (3), motor (4), pen collection apparatus (5) and sterile liquid chamber (6) shown in FIG. 1 belong to the state of the art. The improvement pertaining to our system according to the invention can be seen in the views in FIGS. 2-5.

The conveyor chassis (1) used in the state of the art operates in the manner of an escalator and is intended to provide the circulation of the pens (K), which are dropped on the same, between the surgeon and the nurse. In this way, the pens (K) may be provided with a hygienic state by way of dipping the same in the sterile liquid chambers (6) and may be applied on the patient in a healthy manner. In the system seen in FIG. 1, the band on the conveyor chassis (1) moves at a certain speed and the pens (K) likewise move in the same direction simultaneously with the band. The full (hair follicle-containing) pen (K) and the empty (non-hair follicle-containing) pen (K) move on the same band. Moreover, the pens (K) are fixed on the band by the use of hand. The pen collection apparatus (5) serve the function of preventing the confusion of the empty pens (K) with the full ones.

An improved conveyor assembly (10) for the system according to the invention can be seen in FIG. 3 and FIG. 4. The full and empty pens (K) move on different bands. These bands are separated as the full pen band (10.1) and the empty pen band (10.2). While the full pen band (10.1) moves in the +z direction, the empty pen bands (10.2) move in the −z direction. Owing to the full and empty pens (K) advancing on two different bands, the likelihood of confusing the same is zeroed out despite the possibility for the same to escape the attention of the users. The surgeon to perform the hair transplantation, after taking and using the pens (K) coming via the full pen band (10.1), drops the pens (K) freed from the hair follicles into the empty pen funnel (8). The empty pen funnel (8) is associated with the empty pen band (10.2) wherein the dropped pens (K) are directly fixed on this band. The empty pens (K) advance on the empty pen band (10.2), are received and filled by the nurse, and the full pens (K) are dropped in the full pen funnel (9) and conveyed to the surgeon by the full pen band (10.1).

The robotic arm (11) seen in FIG. 5 performs the hair implantation procedure in a fully automated manner. The robotic arm (11) may perform the operation mechanically on multiple axes by means of the joints (11.1) capable of moving independently of one another. The joints (11.1) are moved by means of the servo motor and are linked together via the female part (11.1.2) and the male part (11.1.1). At least one position sensor is present on the sensitive gripper (11.2). The position sensors to be placed on the head of the patient and the sensors on the sensitive gripper (11.2) will be able to transmit signals to one another and it will be possible to bring the sensitive gripper (11.2) into the proper alignment. This alignment process will be realized by means of special software.

What is claimed is:

1. A direct hair implantation automatic transfer device, comprising:
a conveyor chassis;
a hair implantation pen holder disposed on a band within the conveyor chassis;
a motor providing a movement of the conveyor chassis;
a transparent cover covering the conveyor chassis;
a pen collection apparatus enabling to collect pens;
a sterile liquid chamber enabling to sterilize the pens;
a display showing data about a system and a patient;
a conveyor assembly comprising at least one empty pen band positioned parallel to one another in a direction of z-axis and at least one full pen band positioned between the empty pen bands in a way making a certain angle with respect to the z-axis;
an empty pen funnel and a full pen funnel, wherein lower and upper portions of the empty pen funnel and the full pen funnel are formed by holes and the empty pen funnel and the full pen funnel have a conical or plain cylindrical form with respect to a vertical centerline; and
a robotic arm comprising at least one joint associated in an engaging manner and having a freedom of motion with respect to one another and a sensitive gripper carrying the pen at a tip portion thereof.

2. The direct hair implantation automatic transfer device according to claim 1, wherein the conveyor assembly comprises a full pen band and an empty pen band, wherein the full pen band and the empty pen band move in opposite directions.

3. The direct hair implantation automatic transfer device according to claim 1, wherein the conveyor assembly comprises at least one gear providing support for a movement of the bands.

4. The direct hair implantation automatic transfer device according to claim 1, wherein the conveyor assembly comprises at least one shaft configured to enable a bearing for gears.

5. The direct hair implantation automatic transfer device according to claim 1, wherein the conveyor assembly comprises at least one bearing configured to balance loads imparted on shafts.

6. The direct hair implantation automatic transfer device according to claim 1, further comprising an empty pen funnel, wherein the empty pen funnel is associated with a body in a way to deliver the pens onto the empty pen band.

7. The direct hair implantation automatic transfer device according to claim 1, further comprising a full pen funnel, wherein the full pen funnel is associated with a body in a way to deliver the pens onto the full pen band.

8. The direct hair implantation automatic transfer device according to claim 1, wherein the robotic arm comprises joints configured to enable forward-backward, rightward-leftward, upward-downward and angular movements of the sensitive gripper.

9. The direct hair implantation automatic transfer device according to claim 1, wherein the robotic arm comprises at least one servo motor or step motor providing a movement torque to the joints.

10. The direct hair implantation automatic transfer device according to claim 1, wherein the robotic arm comprises at least one position sensor located on the sensitive gripper.

11. The direct hair implantation automatic transfer device according to claim 8, wherein the joint comprises a male part and a female part providing an engagement-type association.
